(19) United States
(12) Patent Application Publication
(10) Pub. No.: US 2014/0309538 A1
(43) Pub. Date: Oct. 16, 2014

(54) APPARATUS AND METHOD FOR DETECTING PHRENIC NERVE STIMULATION

(71) Applicant: PACESETTER, INC., Sylmar, CA (US)

(72) Inventors: Rohan A. More, Los Angeles, CA (US); Benjamin Coppola, Woodland Hills, CA (US)

(73) Assignee: PACESETTER, INC., Sylmar, CA (US)

(21) Appl. No.: 13/860,351

(22) Filed: Apr. 10, 2013

(51) Int. Cl. A61B 5/00; A61B 5/0205

(52) U.S. Cl. CPC A61B 5/7235; A61B 5/0205; A61B 5/4041
USPC 600/483; 600/300

(57) ABSTRACT

An apparatus and method for detecting phrenic nerve stimulation (PNS) including computing a power spectral density (PSD) of spatial data of a sensor; determining two spectral magnitudes from the PSD; computing a ratio of the two spectral magnitudes; and comparing the ratio to a threshold to detect PNS. In one example, detecting PNS includes computing a PSD of spatial data of an externally placed sensor; determining a diaphragmatic spectral magnitude and a respiratory motion spectral magnitude from the PSD; and computing a ratio of the diaphragmatic spectral magnitude to the respiratory motion spectral magnitude to detect PNS. In one example, detecting PNS includes computing an oscillation amplitude of spatial data of a sensor; computing a baseline wander amplitude of the spatial data of the sensor; computing a ratio of the oscillation amplitude to the baseline wander amplitude; and comparing the ratio to a threshold to detect PNS.

### FIELD

This disclosure relates generally to apparatus and methods for detection of phrenic nerve stimulation.

### BACKGROUND

Phrenic Nerve Stimulation (PNS) occurs in about 10% of all cardiac resynchronization therapy (CRT) patients. It is estimated that nearly 3% of the patients have to be re-operated to correct PNS. To decrease the probability of PNS, it is necessary to bypass pacing vectors that cause PNS. For example, some medical personnel or medical device manufacturers have suggested using multiple pacing configurations to bypass the pacing vector that causes PNS. For example, a pacing lead may provide a multiple quantity of pacing options to the medical personnel (e.g., physician) with the primary goal of picking the best suited pacing option for avoiding phrenic nerve stimulation. Although software-driven workflows have graphically eased the way to test all the offered vector options, there is still no automatic test to check for PNS in all the offered vector options. Thus, a prevalent current practice is for the medical personnel (e.g., physician) to hold his or her hand over the patient's chest and confirm any diaphragm movement as a way of detecting PNS.

### SUMMARY

Disclosed is an apparatus and method for detecting phrenic nerve stimulation. According to one aspect, a method for detecting phrenic nerve stimulation (PNS) including computing a power spectral density (PSD) of spatial data of a sensor; determining a first spectral magnitude and a second spectral magnitude from the power spectral density (PSD); computing a ratio of the first spectral magnitude to the second spectral magnitude; and comparing the ratio to a threshold, wherein if the ratio is equal or greater than the threshold, determining that phrenic nerve stimulation (PNS) is present and wherein if the ratio is less than the threshold, determining that no PNS or minimal PNS is present.

According to one aspect, a device for detecting phrenic nerve stimulation (PNS) comprising a processor and a memory, the memory containing program code executable by the processor for performing the following: computing a power spectral density (PSD) of spatial data of a sensor; determining a first spectral magnitude and a second spectral magnitude from the power spectral density (PSD); computing a ratio of the first spectral magnitude to the second spectral magnitude; and comparing the ratio to a threshold, wherein if the ratio is equal or greater than the threshold, determining that phrenic nerve stimulation (PNS) is present and wherein if the ratio is less than the threshold, determining that no PNS or minimal PNS is present.

According to one aspect, a method for detecting phrenic nerve stimulation (PNS) including computing a power spectral density (PSD) of spatial data of a sensor, wherein the sensor is an external sensor placed on a patient's torso area; determining a diaphragmatic spectral magnitude and a respiratory motion spectral magnitude from the power spectral density (PSD); and computing a ratio of the diaphragmatic spectral magnitude to the respiratory motion spectral magnitude, wherein if the ratio is equal or greater than a threshold, determining that phrenic nerve stimulation (PNS) is present, and if the ratio is less than the threshold, determining that no PNS or minimal PNS is present.

According to one aspect, a device for detecting phrenic nerve stimulation (PNS) comprising a processor and a memory, the memory containing program code executable by the processor for performing the following: computing a power spectral density (PSD) of spatial data of a sensor, wherein the sensor is an external sensor placed on a patient's torso area; determining a diaphragmatic spectral magnitude and a respiratory motion spectral magnitude from the power spectral density (PSD); and computing a ratio of the diaphragmatic spectral magnitude to the respiratory motion spectral magnitude, wherein if the ratio is equal or greater than a threshold, determining that phrenic nerve stimulation (PNS) is present, and if the ratio is less than the threshold, determining that no PNS or minimal PNS is present.

According to one aspect, a method for detecting phrenic nerve stimulation (PNS) including computing an oscillation amplitude of spatial data of a sensor; computing a baseline wander amplitude of the spatial data of the sensor; computing a ratio of the oscillation amplitude to the baseline wander amplitude; and comparing the ratio to a threshold, wherein if the ratio is equal or greater than a threshold, determining that phrenic nerve stimulation (PNS) is present, and wherein if the ratio is less than the threshold, determining that no PNS or minimal PNS is present.

According to one aspect, a device for detecting phrenic nerve stimulation (PNS) comprising a processor and a memory, the memory containing program code executable by the processor for performing the following: computing an oscillation amplitude of spatial data of a sensor; computing a baseline wander amplitude of the spatial data of the sensor; computing a ratio of the oscillation amplitude to the baseline wander amplitude; and comparing the ratio to a threshold, wherein if the ratio is equal or greater than the threshold, determining that phrenic nerve stimulation (PNS) is present, and wherein if the ratio is less than the threshold, determining that no PNS or minimal PNS is present.

According to one aspect, a method for detecting phrenic nerve stimulation (PNS) including obtaining a first respiratory spectral magnitude with no phrenic nerve stimulation (PNS) or minimal PNS is present; defining a threshold based on the first respiratory spectral magnitude; determining a second respiratory spectral magnitude from a power spectral density of spatial data of a sensor; and determining that a phrenic nerve stimulation (PNS) is present if the second respiratory spectral magnitude is less than the threshold or determining that no PNS or minimal PNS is present if the second respiratory spectral magnitude is equal or greater than the threshold.

According to one aspect, a device for detecting phrenic nerve stimulation (PNS) comprising a processor and a memory, the memory containing program code executable by the processor for performing the following: obtaining a first respiratory spectral magnitude with no phrenic nerve stimulation (PNS) or minimal PNS is present; defining a threshold based on the first respiratory spectral magnitude; determining a second respiratory spectral magnitude from a power spectral density of spatial data of a sensor; and determining that a phrenic nerve stimulation (PNS) is present if the second respiratory spectral magnitude is less than the threshold or determining that no PNS or minimal PNS is present if the second respiratory spectral magnitude is equal or greater than the threshold.

Advantages of the present disclosure may include decreasing or minimizing involvement of medical personnel in the detection of phrenic nerve stimulation (PNS) by providing an automated PNS detection procedure, eliminating guess work and reducing human error.

It is understood that other aspects will become readily apparent to those skilled in the art from the following detailed description, wherein it is shown and described various aspects by way of illustration. The drawings and detailed description are to be regarded as illustrative in nature and not as restrictive.

### BRIEF DESCRIPTION OF THE DRAWINGS

FIG. 1 illustrates a first example of displacement versus time of a sensor placed within a coronary sinus during pacing in the X, Y, and Z coordinates.

FIG. 2 illustrates a second example of displacement versus time of a sensor placed within a coronary sinus during pacing in the X, Y, and Z coordinates.

FIG. 3 illustrates an example of a power spectral density versus frequency plot of the X, Y and Z coordinate plots illustrated in FIG. 1.

FIG. 4 illustrates an example of a power spectral density versus frequency plot of the X, Y and Z coordinate plots illustrated in FIG. 2.

FIG. 5 illustrates an example of a communication channel coupled between a sensor system and a programmer system.

FIG. 6 illustrates an example of a patient reference sensor (PRS) and a pacing lead on a patient.

FIG. 7 illustrates a first example flow diagram for detecting phrenic nerve stimulation (PNS).

FIG. 8 illustrates a second example flow diagram for detecting phrenic nerve stimulation (PNS).

FIG. 9 illustrates a third example flow diagram for detecting phrenic nerve stimulation (PNS).

FIG. 10 illustrates a fourth example flow diagram for detecting phrenic nerve stimulation (PNS).

FIG. 11 illustrates an example of a device including a processor in communication with a memory for executing the processes of detecting phrenic nerve stimulation (PNS).

### DETAILED DESCRIPTION

The detailed description set forth below in connection with the appended drawings is intended as a description of various aspects of the present disclosure and is not intended to represent the only aspects in which the present disclosure may be practiced. Each aspect described in this disclosure is provided merely as an example or illustration of the present disclosure, and should not necessarily be construed as preferred or advantageous over other aspects. The detailed description includes specific details for the purpose of providing a thorough understanding of the present disclosure. However, it will be apparent to those skilled in the art that the present disclosure may be practiced without these specific details. In some instances, well-known structures and devices are shown in block diagram form in order to avoid obscuring the concepts of the present disclosure. Acronyms and other descriptive terminology may be used merely for convenience and clarity and are not intended to limit the scope of the present disclosure.

While for purposes of simplicity of explanation, the methodologies are shown and described as a series of acts, it is to be understood and appreciated that the methodologies are not limited by the order of acts, as some acts may, in accordance with one or more aspects, occur in different orders and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with one or more aspects.

Three dimensional (3D) position and orientation information of multiple tools can be collected in real time. Using the X, Y and Z coordinates of a sensor, a plot of displacement over time may be drawn for any given time interval.

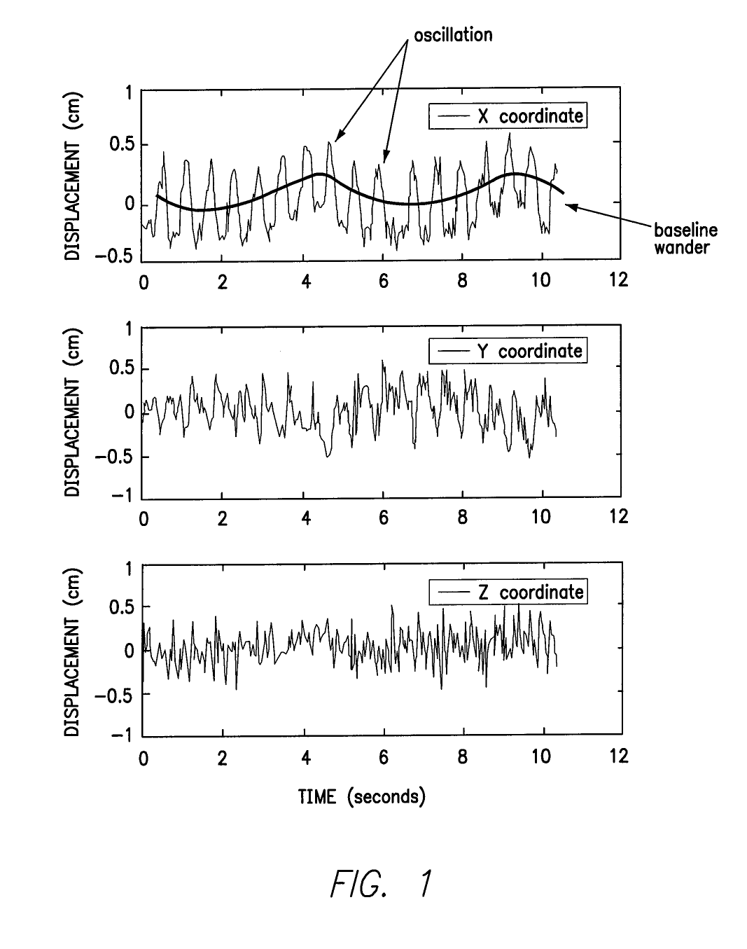

FIG. 1 illustrates a first example of displacement versus time of a sensor placed within a coronary sinus during pacing in the X, Y, and Z coordinates. FIG. 1 shows the X, Y and Z coordinate plots of the same sensor that was placed within the coronary sinus at different time instances. In the example of FIG. 1, the X, Y, and Z coordinates plots of displacement versus time are acquired by placing a pacing lead in one of the coronary sinus branches and paced at 100 beats per minute (bpm). In one example, the pacing lead is a left ventricle (LV), a right ventricle (RV) lead or a multipolar lead. One skilled in the art would understand that other types of pacing lead or other pacing tools may be used without affecting the scope and spirit of the present disclosure. In the time series data of FIG. 1, periodicity may be observed in the displacement signal from the sensor resulting from both cardiac motion (oscillations) and respiratory motion (baseline wander) shown in the X-coordinate plot. In FIG. 1, the baseline wander in the Y and Z coordinate plots are not drawn.

The oscillation characteristic of the X, Y and Z coordinate plots shown in FIG. 1 oscillates between the displacement interval of 0.5 cm to −0.5 cm. The baseline wander of the X, Y and Z coordinate plots shown in FIG. 1 appears to have a slight sinusoidal shape over time. The sinusoidal shape of the baseline wander of the X, Y, and Z coordinates plots shown indicates that there is no or minimal phrenic nerve stimulation.

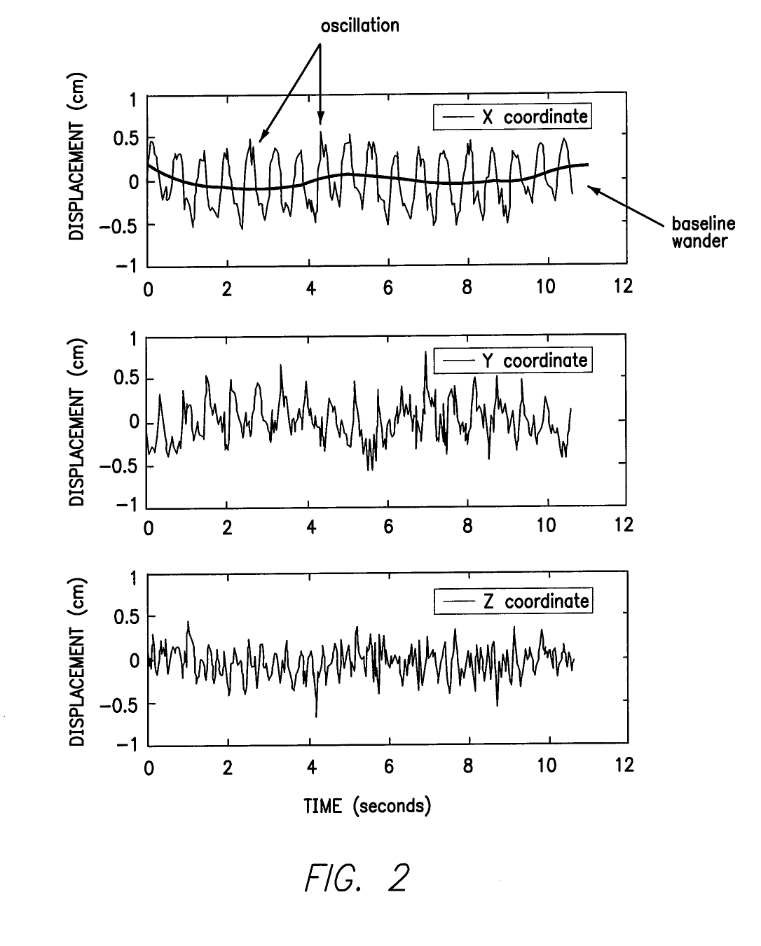

FIG. 2 illustrates a second example of displacement versus time of a sensor placed within a coronary sinus during pacing in the X, Y, and Z coordinates. The X, Y and Z coordinate plots shown in FIG. 2 were acquired when the LV lead was placed in a different branch of the coronary sinus and paced at the same heart rate. In FIG. 2, although the oscillation characteristic of the X, Y and Z coordinate plots appears to be similar to the oscillation characteristic of the X, Y and Z coordinate plots shown in FIG. 1, the baseline wander in the X, Y and Z coordinate plots shown in FIG. 2 is flat. In the example of FIG. 2, only the baseline wander in the X coordinate plot is drawn. The flat shape of the baseline wander of the X, Y, and Z coordinates plots shown indicates that there is phrenic nerve stimulation due to pacing. The data shown in FIGS. 1 and 2 are collected from canine studies.

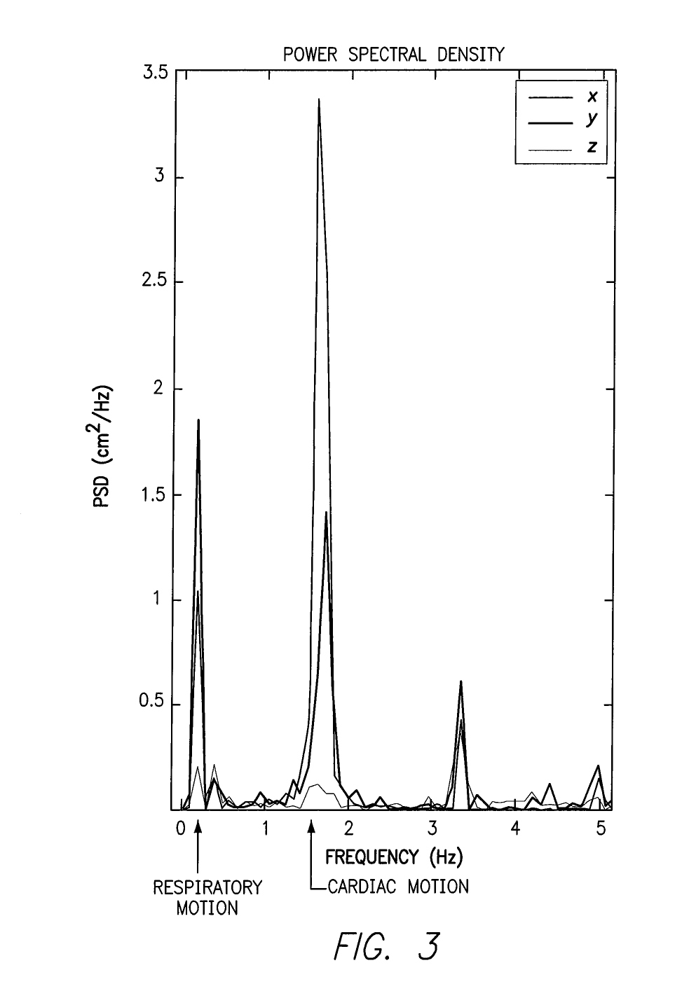

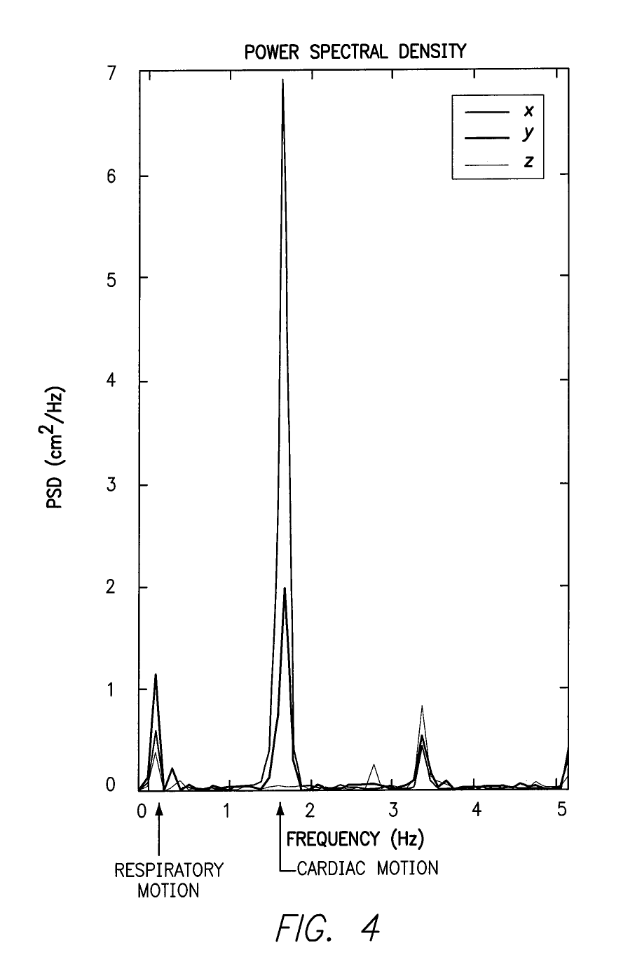

FIG. 3 illustrates an example of a power spectral density versus frequency plot of the X, Y and Z coordinate plots illustrated in FIG. 1. FIG. 4 illustrates an example of a power spectral density versus frequency plot of the X, Y and Z coordinate plots illustrated in FIG. 2. In the examples of FIGS. 3 and 4, the peak spectral magnitude between 0 and 0.5 Hz represents the respiratory motion. And, the peak spectral magnitude between 1.5 and 2 Hz represents the cardiac motion (100 bpm=1.67 Hz). When PNS occurs as a result of pacing, the diaphragm contracts forcefully and so normal respiratory motion is interrupted. This is manifested in the power spectral density (PSD). The ratio of cardiac motion spectral magnitude to respiratory motion spectral magnitude increases. This can be seen in FIG. 3. For the example in FIG. 3, using the X coordinate, the ratio of cardiac motion spectral magnitude to respiratory motion spectral magnitude increases from 3.2 to 11.8. The power spectral density (PSD) plot shown in FIG. 3 indicates that there is no or minimal phrenic nerve stimulation (PNS). On the other hand, the power spectral density (PSD) plot shown in FIG. 4 indicates that there is phrenic nerve stimulation (PNS).

In one example, real time tracking of the sensor within a coronary sinus branch during pacing allows computing the cardiac motion spectral magnitude to respiratory motion spectral magnitude ratio just by using a few cardiac cycles. Selecting the right coordinate axis that captures the major component of respiratory motion may help compute the ratio more accurately. Since more than one power spectral density (PSD) may be required for comparison, multiple pacing configurations may be advantageous to determine the lower cardiac motion spectral magnitude to respiratory motion spectral magnitude ratio. In one example, the lowest ratio may be deemed as normal pacing with no phrenic nerve stimulation (PNS). In the example of a single vector detection, a non-PNS graph may be computed by using an right ventricle (RV) pacing configuration as the baseline, that is, as having no phrenic nerve stimulation (PNS).

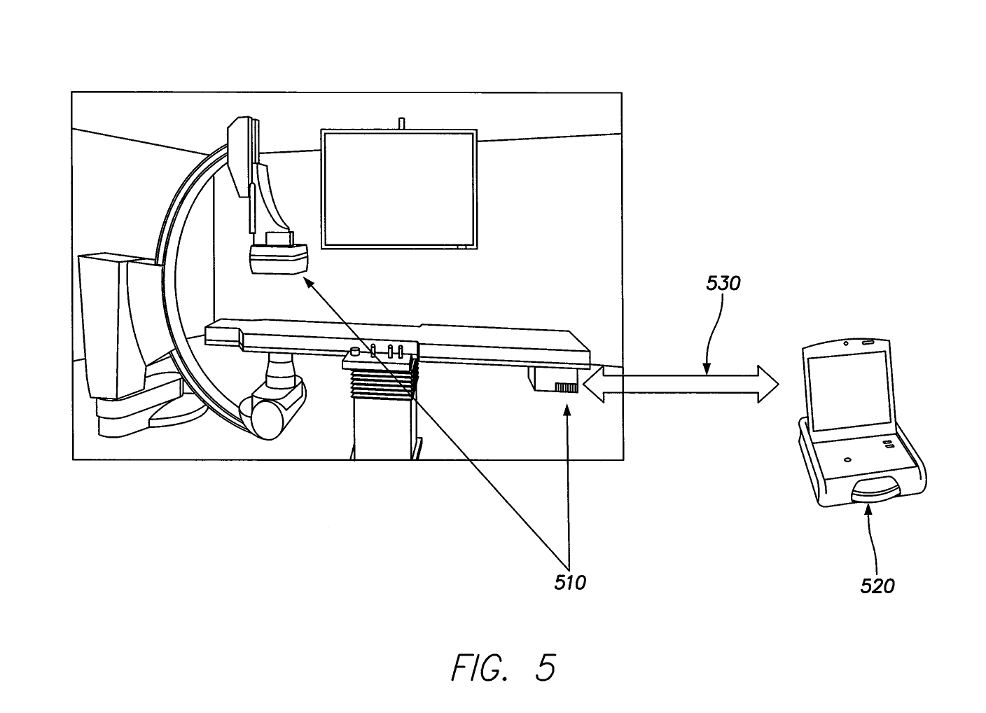

FIG. 5 illustrates an example of a communication channel 530 coupled between a sensor system 510 and a programmer system 520. As shown in FIG. 5, the sensor system may include multiple components coupled to perform a sensing function. The close proximity of the sensor system and the programmer system in, for example, a cardiac electrophysiology (EP) room may facilitate communication exchange between the two. In one example, the communication channel between the sensor system and the programmer system communicates detection of phrenic nerve stimulation (PNS) using the position and orientation of sensor(s) monitoring the patient in real-time. By tracking the displacement of the sensor, the programmer system computes the power spectrum density (PSD) of the displacement of the sensor over time. The PNS data may then be used to detect phrenic nerve stimulation (PNS). In addition to being a wired channel between the sensor system and the programmer system, in one example, the communication channel is a wireless channel.

For example, once a pacing lead is in place within a patient and the condition is ready for pacing vector selection, the programmer paces through all pacing vectors sequentially with a selected pacing amplitude (e.g., a 10V pacing amplitude). For every single vector, the sensor system collects the 3D coordinates (i.e., X, Y and Z coordinates) of the sensor that is placed within the patient (e.g., in a patient's heart vessel). In one example, the sensor is associated with an enabled guidewire, an inner catheter or an outer catheter, any of which may be used during a cardiac resynchronization therapy (CRT) implant. Using the cardiac motion spectral magnitude to respiratory motion spectral magnitude ratio, the sensor system detects event of PNS and reports back to the programmer system via communication channel coupled between the two systems. In one example, the programmer system changes the pacing vector and applies a pulse (e.g., 10V pulse) to the new vector. This process is repeated for all the pacing vectors. At the end of the test, the programmer system generates a list of vectors that are marked with ‘PNS Detected’. In one example, the programmer system marks each of the pacing vectors with which PNS is detected. The vectors marked with ‘PNS Detected” are the pacing vectors with which PNS is detected.

In one example the process of automatic PNS detection may be incorporated with automatic capture threshold algorithm used on the implantable device programmer system. In one example, PNS may be detected by the stand-alone sensor system without communication with the programmer system based on the inputs from various sensors including a Patient Reference Sensor (PNS).

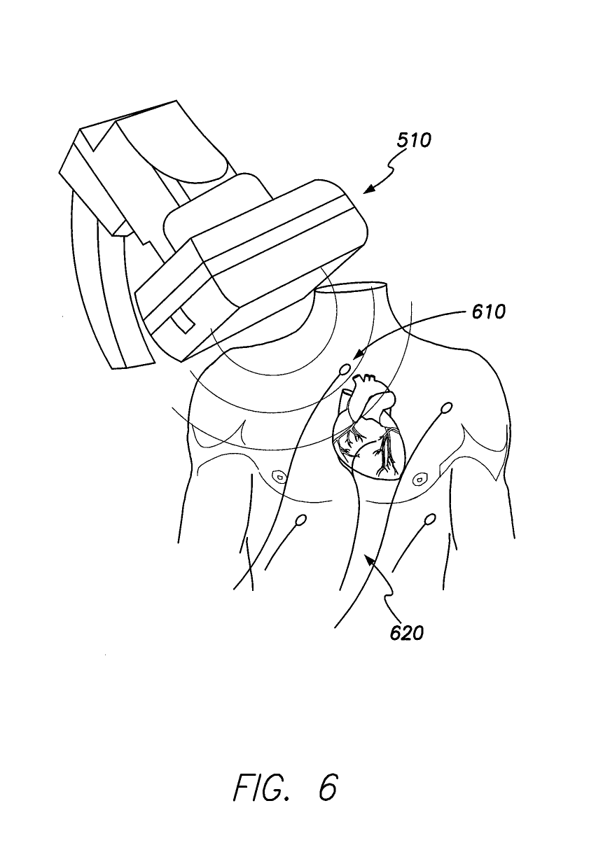

FIG. 6 illustrates an example of a patient reference sensor (PRS) 610 and a pacing lead 620 on a patient. In one example, the patient reference sensor (PRS) 610 is part of the sensor system 510. In one example, the pacing lead is a left ventricle (LV) lead. In another example, the pacing lead is a right ventricle (RV) lead. The patient reference sensor (PRS) is placed externally on the patient's torso area to detect respiratory motion and diaphragmatic stimulation. In one example, the torso area is the chest area of the patient. PRS has the capability to sense respiratory motion in the order of millimeters. In one example, an algorithm is used to distinguish between rhythmic respiratory motion and diaphragmatic stimulation by measuring their amplitude ratio in a power density spectrum versus frequency plot. For example, the occurrence of diaphragmatic stimulation is detected to identify phrenic nerve stimulation (PNS) associated with a particular pacing vector.

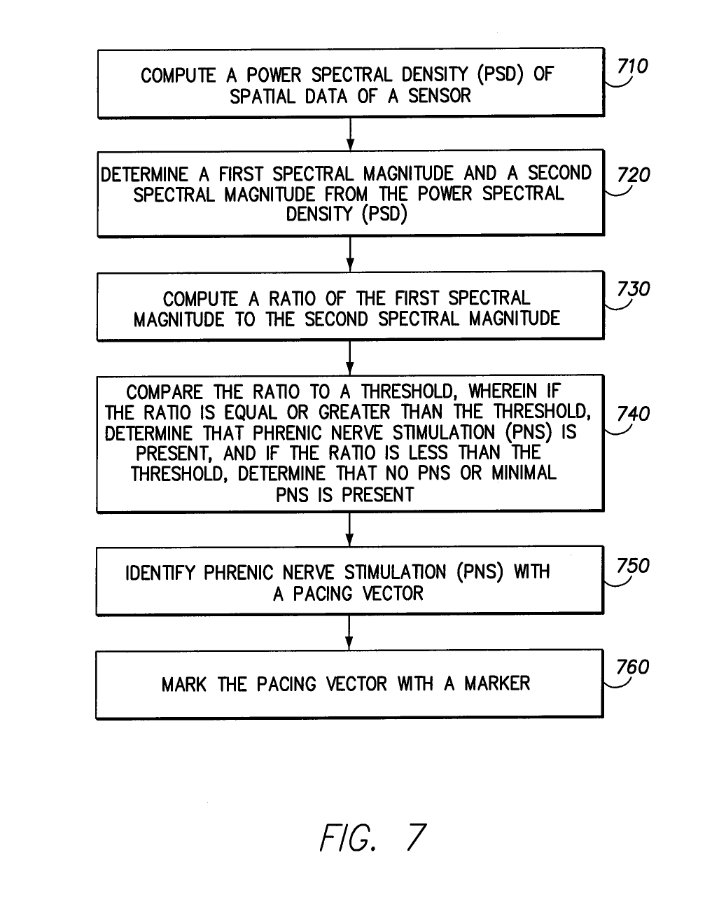

FIG. 7 illustrates a first example flow diagram for detecting phrenic nerve stimulation (PNS). In block 710, compute a power spectral density (PSD) of spatial data of a sensor. In one example, the spatial data includes one or more of the following: position data or orientation data of the sensor over time. In one example, the position data includes 3 dimensions (3D). In one example, the orientation data includes 1, 2 or 3 degrees of freedom. In one example, the step of “computing a power spectral density of spatial data of a sensor” includes taking a Fourier transform of the spatial data. This may also include taking the magnitude squared of the Fourier transform. In one example, the step of “computing a power spectral density of spatial data of a sensor” includes using parametric spectral estimation of the spatial data. In another example, the step of “computing a power spectral density of spatial data of a sensor” includes using non-parametric spectral estimation of the spatial data. In one example, the step of “computing a power spectral density of spatial data of a sensor” includes taking a wavelet transform of the spatial data. In one example, the step of “computing a power spectral density of spatial data of a sensor” includes taking a periodogram of the spatial data. This may include weighting the periodogram with a predetermined weighting function. In one example, the step of “computing a power spectral density of spatial data of a sensor” includes taking a periodogram of a moving average of the spatial data. Although various examples of computing the PSD are disclosed herein, one skilled in the art would understand that the examples disclosed herein are not exclusive and that other examples for computing the PSD may be used without affecting the scope and spirit of the present disclosure.

In one example, the sensor is embedded in a chest cavity. In another example, the sensor is embedded within a heart tissue. In one example, the sensor is embedded within a blood vessel. One example of the blood vessel is a coronary sinus branch. In yet another example, the sensor may be placed externally on a patient, such as, on a patient's torso area which may be on the chest area. In another example, the sensor is placed on the patient's back. One skilled in the art would understand that the location of the initial placement of the sensor may vary according to various factors, such as but not limited to, medical personnel's choice, patient's medical condition, the type or sensor used, etc., without affecting the scope and spirit of the present disclosure. Additionally, one skilled in the art would understand that the examples of the sensor's initial location disclosed herein are not exclusive.

In one example, the sensor is one of the following: an electromagnetic sensor, a ultrasonic sensor, an acoustic sensor, an impedance-based sensor, etc. In one example, the sensor is a set of multiple sensors of one of the following type of sensors: electromagnetic sensors, ultrasonic sensors or acoustic sensors impedance-based sensor. In one example, the multiple sensor set includes more than one type of sensors in its set. In one example, one or more sensors of the multiple sensor set is placed internally in the patient while another one or more sensors the multiple sensor set is placed externally on the patient. For example, a sensor may be placed within a branch of the coronary sinus, a sensor may be placed on the torso area, a sensor may be placed on the patient's back area, or combination thereof may be used.

Other types of sensors or combination of two or more of the types of sensors disclosed herein may be used. In one example, for the sensor placed internally in a patient, the sensor is associated with one of the following: a guidewire, a catheter, a lead, a stylet, etc.

In block 720, determine a first spectral magnitude and a second spectral magnitude from the power spectral density (PSD). In one example, the first spectral magnitude is a cardiac motion spectral magnitude, and the second spectral magnitude is a respiratory motion spectral magnitude. In another example, both the first and second spectral magnitude relate to a cardiac signal. In yet another example, both the first and second spectral magnitude relate to a respiratory signal. In one example, the cardiac motion spectral magnitude occurs at a higher frequency than the respiratory motion spectral magnitude. For example, the cardiac motion spectral magnitude may occur at a frequency between 1.5 to 3 Hz. And, for example, the respiratory motion spectral magnitude may occur at a frequency between 0 to 0.5 Hz.

In block 730, compute a ratio of the first spectral magnitude to the second spectral magnitude. Following block 730, in block 740, compare the ratio to a threshold. In one example, if the ratio is equal or greater than the threshold, determine that phrenic nerve stimulation (PNS) is present. And, if the ratio is less than the threshold, determine that no PNS or minimal PNS is present. In one example, the value of the threshold is derived from a right ventricle (RV) pacing configuration. In block 750, identify the PNS with a pacing vector. In one example, the pacing vector is a combination of a cathode and an anode configuration. In block 760, mark the pacing vector with a marker. In one example, the pacing vector is executed by a programmer system. In one example, the sensor is part of a sensor system, and a communication channel couples the sensor system with the programmer system so the two systems can communicate with each other. In one example, the programmer system marks the pacing vector with a marker to indicate that PNS was detected with that pacing vector.

In one aspect, the steps illustrated in FIG. 7 are repeated for a plurality of pacing vectors where each of the plurality of pacing vectors is different. In one example, each of the plurality of pacing vectors has a different pacing voltage. In addition, a plurality of ratios of cardiac motion spectral magnitude to the respiratory motion spectral magnitude is generated for detecting PNS. In this example, each of the plurality of ratios corresponds to one of the plurality of pacing vectors. And, in one example, use the lowest ratio from the plurality of ratios as a baseline for indicating the absence of phrenic nerve stimulation (PNS).

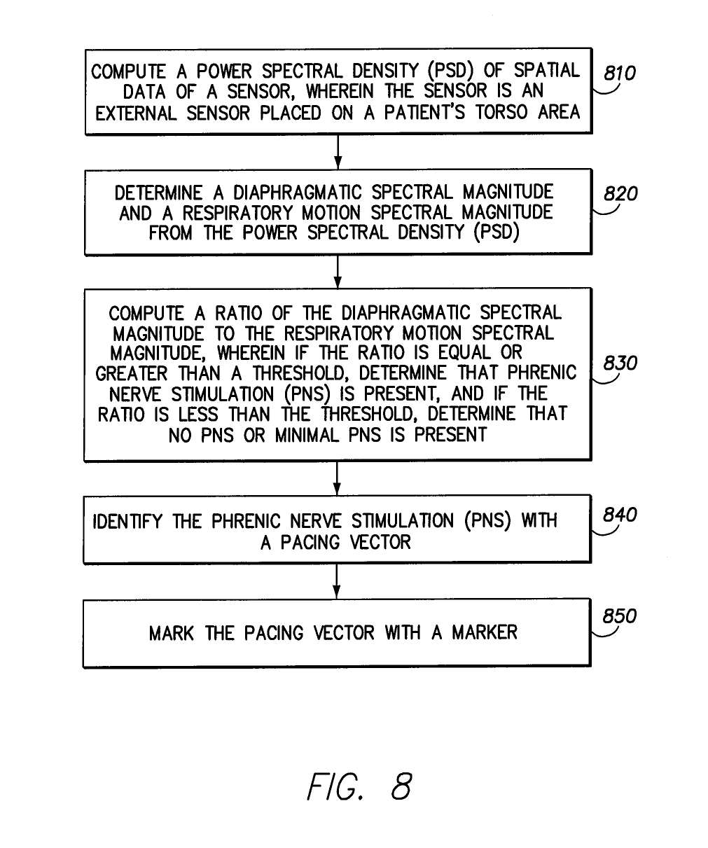

FIG. 8 illustrates a second example flow diagram for detecting phrenic nerve stimulation (PNS). In block 810, compute a power spectral density (PSD) of spatial data of a sensor, wherein the sensor is an external sensor placed on a patient's torso area. In one example, the torso area is the patient's chest area. In one example, the spatial data includes as least one dimension of position data of the sensor.

The various ways of computing PSD as described with the example of FIG. 7 are equally applicable to the example of FIG. 8. In one example, the sensor is one of the following: an electromagnetic sensor, a ultrasonic sensor, an acoustic sensor, an impedance-based sensor, etc. In one example, the sensor is a set of multiple sensors which may include one or more types of sensors. In one example, one of the multiple sensors is placed on the patient's torso area while another of the multiple sensors is placed on the patient's back.

In block 820, determine a diaphragmatic spectral magnitude and a respiratory motion spectral magnitude from the power spectral density (PSD). In block 830, compute a ratio of the diaphragmatic spectral magnitude to the respiratory motion spectral magnitude. In one example, if the ratio is equal or greater than a threshold, determine that phrenic nerve stimulation (PNS) is present. And, if the ratio is less than the threshold, determine that no PNS or minimal PNS is present. In one example, the value of the threshold is derived from a right ventricle (RV) pacing configuration. In block 840, identify the PNS with a pacing vector. In block 850, mark the pacing vector with a marker. In one example, the sensor is part of a sensor system which is coupled to a programmer system by a communication channel. In one example, the programmer system marks the pacing vector with a marker to indicate that that pacing vector is associated with a PNS.

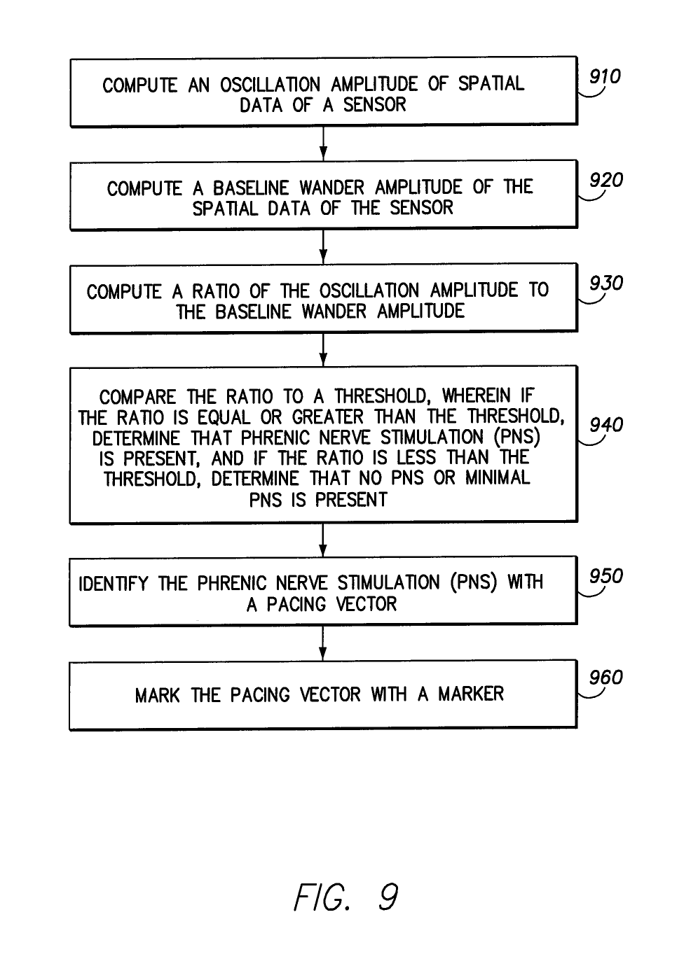

FIG. 9 illustrates a third example flow diagram for detecting phrenic nerve stimulation (PNS). In block 910, compute an oscillation amplitude of spatial data of a sensor. In one example, the sensor is one of the following: an electromagnetic sensor, a ultrasonic sensor, an acoustic sensor, an impedance-based sensor, etc. In one example, the sensor is a set of multiple sensors which may include one or more types of sensors. In one example, the spatial data includes as least one dimension of position data of the sensor. In block 920, compute a baseline wander amplitude of the spatial data of the sensor. In block 930, compute a ratio of the oscillation amplitude to the baseline wander amplitude. In block 940, compare the ratio to a threshold. In one example, if the ratio is equal or greater than the threshold, determine that phrenic nerve stimulation (PNS) is present. And, if the ratio is less than the threshold, determine that no PNS or minimal PNS is present. In one example, the value of the threshold is derived from a right ventricle (RV) pacing configuration. In block 950, identify the PNS with a pacing vector. In block 960, mark the pacing vector with a marker. In one example, the sensor is part of a sensor system which is coupled to a programmer system by a communication channel. In one example, the programmer system marks the pacing vector with a marker to indicate that that pacing vector is associated with a PNS.

In one example, one or more sensors of the multiple sensor set is placed internally in the patient while another one or more sensors the multiple sensor set is placed externally on the patient. For example, a sensor may be placed within a branch of the coronary sinus, a sensor may be placed on the torso area, a sensor may be placed on the patient's back area, or combination thereof may be used. In one example, for the sensor placed internally in a patient, the sensor is associated with one of the following: a guidewire, a catheter, a lead, a stylet, etc.

In one aspect, phrenic nerve stimulation (PNS) causes the diaphragm to contract and therefore prevent normal breathing, thereby limiting respiratory motion and concomitantly decreasing the respiratory spectral magnitude. The change in cardiac spectral magnitude may be secondary to this breathing effect. The contraction of the diaphragm causes inspiration, which reduces thoracic pressure, and thus, may reduce the resistance to cardiac motion.

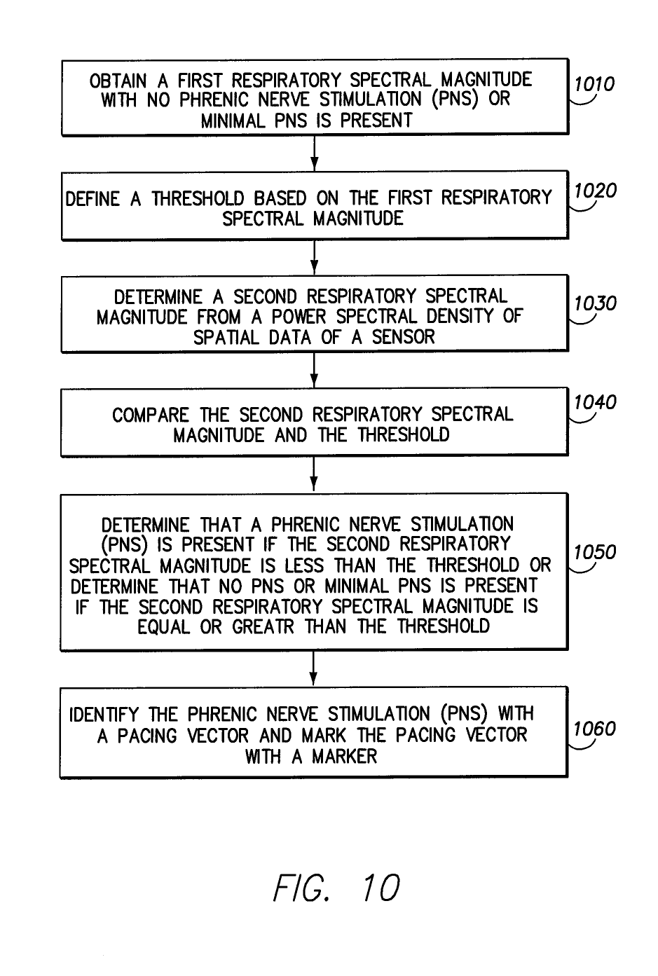

FIG. 10 illustrates a fourth example flow diagram for detecting phrenic nerve stimulation (PNS). In block 1010, obtain a first respiratory spectral magnitude with no phrenic nerve stimulation (PNS) or minimal PNS is present. In one example, the first respiratory spectral magnitude is obtained during a right ventricle (RV) pacing. In one example, the first respiratory spectral magnitude is obtained during a right atrium (RA) pacing. In yet another example, the first respiratory spectral magnitude is obtained when there is no pacing. In one example, obtain the first respiratory spectral magnitude of the right ventricle pacing by measurement. In another example, obtain the first respiratory spectral magnitude by simulation. In yet another example, obtain the first respiratory spectral magnitude by analysis. One skilled in the art would understand that there are many techniques to obtain a respiratory spectral magnitude, that the examples disclosed herein are not exclusive, and that other techniques not disclosed herein may be equally applicable within the scope and spirit of the present disclosure.

In block 1020, define a threshold based on the first respiratory spectral magnitude. The threshold is used to indicate no phrenic nerve stimulation (no PNS). In block 1030, determine a second respiratory spectral magnitude from a power spectral density of spatial data of a sensor. In one example, the first and second respiratory spectral magnitudes are centered at a same frequency.

In one example, the spatial data includes one or more of the following: position data of the sensor over time or orientation data of the sensor over time. In one example, the sensor is one of the following: an electromagnetic sensor, a ultrasonic sensor, an acoustic sensor or an impedance-based sensor. In one example, the sensor is placed internally in a patient, for example, in a chest cavity, a heart tissue, a blood vessel, or a coronary sinus branch. In another example, the sensor is placed externally on a patient's torso area. The sensor may be a set of multiple sensors.

In one example, the pacing configurations for the first respiratory spectral magnitude and the second respiratory spectral magnitude are different. In one example, the power spectral density is based on a left ventricle (LV) pacing configuration. In one example the power spectral density (PSD) is computed by one of the following: taking a Fourier transform of the spatial data and/or taking the magnitude squared of the Fourier transform, using parametric spectral estimation of the spatial data; using non-parametric spectral estimation of the spatial data; taking a wavelet transform of the spatial data; taking a periodogram of the spatial data; or taking a periodogram of a moving average of the spatial data.

In block 1040, compare the second respiratory spectral magnitude and the threshold. In block 1050, determine that a phrenic nerve stimulation (PNS) is present if the second respiratory spectral magnitude is less than the threshold or determine that no PNS or minimal PNS is present if the second respiratory spectral magnitude is equal or greater than the threshold. In block 1060, identify the phrenic nerve stimulation (PNS) with a pacing vector and mark the pacing vector with a marker. In one example, repeat the steps of FIG. 10 for a plurality of pacing vectors, wherein each of the plurality of pacing vectors is different.

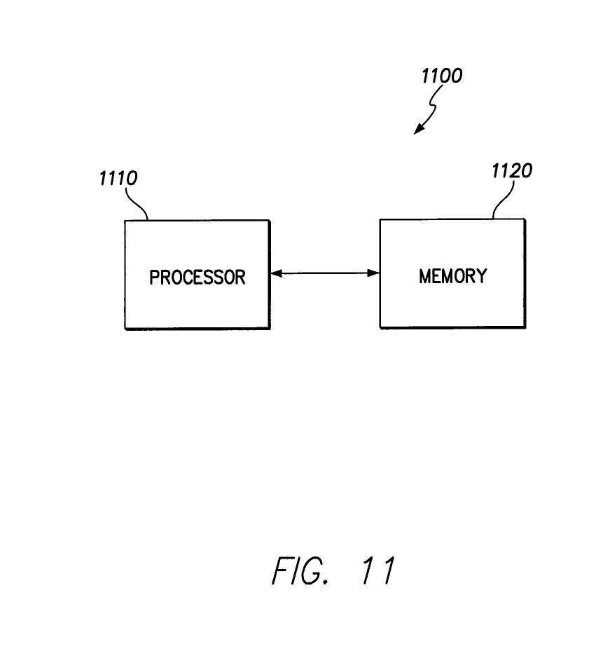

FIG. 11 illustrates an example of a device 1100 including a processor 1010 in communication with a memory 1120 for executing the processes of detecting phrenic nerve stimulation (PNS). In one example, the device 1100 is used to implement the algorithms illustrated in FIGS. 7, 8, 9 and 10. In one aspect, the memory 1120 is located within the processor 1110. In another aspect, the memory 1120 is external to the processor 1110. In one aspect, the processor includes circuitry for implementing or performing the various flow diagrams, logical blocks and/or modules described herein.

While for purposes of simplicity of explanation, the methodologies are shown and described as a series of acts, it is to be understood and appreciated that the methodologies are not limited by the order of acts, as some acts may, in accordance with one or more aspects, occur in different orders and/or concurrently with other acts from that shown and described herein. For example, those skilled in the art will understand and appreciate that a methodology could alternatively be represented as a series of interrelated states or events. Moreover, not all illustrated acts may be required to implement a methodology in accordance with one or more aspects.

The previous description of the disclosed aspects is provided to enable any person skilled in the art to make or use the present disclosure. Various modifications to these aspects will be readily apparent to those skilled in the art, and the generic principles defined herein may be applied to other aspects without departing from the spirit or scope of the disclosure.

1. A method for detecting phrenic nerve stimulation (PNS) comprising:
computing a power spectral density (PSD) of spatial data of a sensor;
determining a first spectral magnitude and a second spectral magnitude from the power spectral density (PSD);
computing a ratio of the first spectral magnitude to the second spectral magnitude; and
comparing the ratio to a threshold, wherein if the ratio is equal or greater than the threshold, determining that phrenic nerve stimulation (PNS) is present, and wherein if the ratio is less than the threshold, determining that no PNS or minimal PNS is present.

2. The method of claim 1, wherein the spatial data includes one or more of the following: position data of the sensor over time or orientation data of the sensor over time.

3. The method of claim 1, further comprising computing the power spectral density (PSD) by taking a Fourier transform of the spatial data.

4. The method of claim 4, further comprising taking a magnitude squared of the Fourier transform.

5. The method of claim 1, further comprising computing the power spectral density (PSD) by one of the following: using parametric spectral estimation of the spatial data; using non-parametric spectral estimation of the spatial data; taking a wavelet transform of the spatial data; taking a periodogram of the spatial data; or taking a periodogram of a moving average of the spatial data.

6. The method of claim 1, wherein the sensor is one of the following: an electromagnetic sensor, a ultrasonic sensor, an acoustic sensor or an impedance-based sensor.

7. The method of claim 6, wherein the sensor is placed internally in a patient.

8. The method of claim 7, wherein the sensor is placed within one of the following locations of the patient: a chest cavity, a heart tissue or a blood vessel.

9. The method of claim 7, wherein the sensor is placed within a coronary sinus branch.

10. The method of claim 6, wherein the sensor is placed externally on a patient's torso area.

11. The method of claim 1, wherein the sensor comprises a set of multiple sensors.

12. The method of claim 1, wherein the first spectral magnitude is a cardiac motion spectral magnitude, and the second spectral magnitude is a respiratory motion spectral magnitude.

13. The method of claim 12, wherein the threshold is derived from a right ventricle (RV) pacing configuration.

14. The method of claim 1, further comprising identifying the phrenic nerve stimulation (PNS) with a pacing vector and marking the pacing vector with a marker.

15. The method of claim 1, further comprising repeating the steps of claim 1 for a plurality of pacing vectors, wherein each of the plurality of pacing vectors is different.

16. A device for detecting phrenic nerve stimulation (PNS) comprising a processor and a memory, the memory containing program code executable by the processor for performing the following:
computing a power spectral density (PSD) of spatial data of a sensor;
determining a first spectral magnitude and a second spectral magnitude from the power spectral density (PSD);
computing a ratio of the first spectral magnitude to the second spectral magnitude; and
comparing the ratio to a threshold, wherein if the ratio is equal or greater than the threshold, determining that phrenic nerve stimulation (PNS) is present and wherein if the ratio is less than the threshold, determining that no PNS or minimal PNS is present.

17. The device of claim 16, wherein the first spectral magnitude is a cardiac motion spectral magnitude, and the second spectral magnitude is a respiratory motion spectral magnitude.

18. The device of claim 16, wherein the memory further comprising program code for identifying the phrenic nerve stimulation (PNS) with a pacing vector and marking the pacing vector with a marker.

19. The device of claim 16, wherein the device comprises a sensor system, a programmer system and a communication channel coupling the sensor system and the programmer system, and wherein the sensor is part of the sensor system.

20. A method for detecting phrenic nerve stimulation (PNS) comprising:
computing an oscillation amplitude of spatial data of a sensor;
computing a baseline wander amplitude of the spatial data of the sensor;
computing a ratio of the oscillation amplitude to the baseline wander amplitude; and
comparing the ratio to a threshold, wherein if the ratio is equal or greater than the threshold, determining that phrenic nerve stimulation (PNS) is present, and wherein if the ratio is less than the threshold, determining that no PNS or minimal PNS is present.
